(19) United States
(12) Patent Application Publication
(10) Pub. No.: US 2008/0228033 A1
(43) Pub. Date: Sep. 18, 2008

(54) Optical Coherence Tomography Probe Device

(75) Inventors: Alexandre R. Tumlinson, Tucson, AZ (US); Jennifer K. Barton, Tucson, AZ (US); Wolfgang Drexler, Vienna (AT); Boris Povazay, Vienna (AT)

Correspondence Address: LERNER GREENBERG STEMER LLP, P O BOX 2480, HOLLYWOOD, FL, 33022-2480, US

(73) Assignees: MEDIZINISCHE UNIVERSITÄT WIEN, Vienna (AT); THE ARIZONA BOARD OF REGENTS, Tucson, AZ (US)

(21) Appl. No.: 11/994,713

(22) PCT Filed: Jun. 30, 2006
(86) PCT No.: PCT/AT2006/000277

§ 371 Date: Apr. 2, 2008

(30) Foreign Application Priority Data
Jul. 4, 2005 (AT) A 1118/2005

(51) Int. Cl. A61B 1/04

(52) U.S. Cl. 600/112

(57) ABSTRACT

Optical coherence tomograph (OCT) probe device (1) comprising an endoscope (6) which is adapted to be coupled to a light source (2) and has a distal tip portion (6.2), the tip portion (6.2) including focussing lens means (11) and a window (5) for directing light to a subject (7) to be scanned, and for receiving light scattered at the subject (7), to send the scattered light back through the endoscope (6) so that it may be applied to a detector (3) together with reference light, said OCT probe device (1) comprising a beam splitter (13; 17) to separate said reference light from the remaining light, as well as a reference light reflector (4; 20) for reflecting the reference light back so that it is composed to the light returned from the subject (7); the beam splitter (13; 17) and the reference light reflector (4; 20) are located in the tip portion (6.2) of the endoscope means (6) behind the focussing lens means (11) through which the composed light is sent back.

The present invention relates to an optical coherence tomography (OCT) probe device comprising
an endoscope means which has a first, proximal portion which is adapted to be coupled to a light source through optical coupling means, as well as a second, distal tip portion,
said endoscope means defining a light path in its interior to send light emitted from the light source and coupled into the proximal portion to the tip portion,
the tip portion including focussing lens means and a window for directing light to a subject to be scanned, and for receiving light scattered at the subject, to send the scattered light back through the endoscope means to the optical coupling means which are further adapted to apply the scattered light together with reference light to a detector,
said OCT probe device further comprising a beam splitter to separate reference light from the remaining light used to be scattered at the subject, as well as a reference light reflector for reflecting the reference light back so that it is composed to the light returned from the subject, to obtain an interference signal for the detector.

From U.S. Pat. No. 6,564,089 B2, e.g., it is known to use optical imaging devices in the form of OCT (Optical Coherence Tomography) endoscope devices to scan subjects, as biological tissues, e.g. blood vessels, to obtain a high resolution tomogram of the inside of such tissues on the basis of low-coherent interference with scattered light from the tissue. Usually, the light of a low-coherence light source is coupled into an endoscope arm as well as into a reference arm arranged “in parallel” to the endoscope arm by means of an optical beam splitter. Light sent back from both arms is transmitted then by the optical coupler to a detector which detects the resulting interference signal.

Clinical application OCT endoscopy requires that the endoscope probes be replaceable without engineering intervention. Since uncompensated inter-endoscope pathlength differences of less than 1 mm adversely affect performance of conventional Michelson interferometer-based OCT devices, designs insensitive to endoscope pathlength are desirable. Thus, it has been intended e.g. to use a topology including a fiber stretching autocorrelator with Faraday mirrors to enable interchange of probes without a predefined probe length or compensation. Ultra high resolution OCT endoscopy presents a special challenge because dispersion and polarization matching between the signal and reference arms of the OCT device must be performed over a wide spectral bandwidth that usually involves a special combination of multiple materials with different dispersion profiles. Numerical methods for compensating dispersion are computationally expensive, suffer from acquisition noise, and if used, will perform best when the real dispersion mismatch is already well compensated in the system.

Frequency domain OCT (FD-OCT) operates on the principle that light combined from a sample scatterer and a reference mirror interfere to form a pattern that is dependant on the difference in path length between the sample scatterer and the reference mirror. If the scatterer and reference are nearly the same optical paths from the beamsplitter, the interference pattern is a low frequency modulation across the optical spectrum. If the optical path difference between the scatterer and reference from the beamsplitter is large, the interference pattern is a high frequency modulation across the optical spectrum. If the source emits a broad bandwidth of light, this modulated optical spectrum can be detected simultaneously with a spectrometer. If the source has a scanning wavelength emission, this modulated optical spectrum is encoded in time. In either case the original spatial distribution of many scatterers can be discertained by a simple Fourier transform of the modulated optical spectrum. The reference mirror must be placed at a nearly equal pathlength to the scattering object of interest to allow a detectable frequency of modulation on the optical spectrum.

FD-OCT simultaneously provides a signal-to-noise (SNR) advantage over the traditional time domain method (TD-OCT) and requires no moving parts in the reference arm, as are present in the device of U.S. Pat. No. 6,564,089 B2. FD-OCT devices without moving depth scanning components allow imaging at rates more than one order of magnitude higher than was previously possible and is responsible for the rapid development of OCT endoscopy. Faster imaging with FD-OCT enables to take endoscopic tomograms with reduced motion artifacts.

On the other hand, from A. B. Vakhtin, D. J. Kane, W. R. Wood, and K. A. Peterson, “Common-path interferometer for frequency-domain optical coherence tomography”, Applied Optics 42, 6953-6958 (2003), it has become known in a freespace FD-OCT interferometer device that the static reference arm can be replaced with a “common path” approach where the reference reflection comes from a surface within the path to the sample rather than from a physically separated reference arm. For example, a glass slide in contact with the sample can act as a beam splitter, and the reference reflector simultaneously. Since the reflectivity of this beam splitting surface is not only influencing the overall reflectivity in the reference arm, but also intrinsically changes the amount of light that is sent back from the sample, the freedom to select a specific splitting ratio and reference reflectivity is limited.

A comparable concept, without a separate reference arm extending from a beam splitting optical coupler in parallel to a signal light arm, is shown in P. Koch, G. Huettmann, D. Boller, J. Weltzel, and E. Koch, “Ultra high resolution Fourier domain OCT in dermatology,” presented at the Coherence domain optical methods and optical coherence tomography in biomedicine IX, San Jose, Calif., USA, 23-26 Jan. 2005; here, a handheld skin probe is presented that uses a fiber coupled light source and a detector, but uses a distal free space interferometer to reduce sensitivity to system vibration and fiber induced polarization and dispersion mismatch. However, this design lacks a compact structure, and this the more since both in the signal light arm and in the reference arm, focussing optics are located distally behind the beam splitter.

Backreflections occurring at the inside surface of an endoscope window element are usually considered a nuisance to be suppressed, and it has been proposed to coat window surfaces, to insert index matching fluid, and to use an off-normal beam exit angle, to possibly solve this problem.

It is now an object of this invention to provide an OCT probe device comprising an endoscope means which is simple in structure and inexpensive but yet highly efficient in operation.

It is a further object of the invention to provide an OCT probe device that has an improved imaging performance, and in particular allows to achieve ultra high resolution OCT application.

According to still another object, it is intended to provide an OCT probe device without compensation problems when a specific endoscope present in the device is to be replaced with another endoscope, and with a “plug-and-play” feature when starting operation of the device demanded by hygienic safety standards.

In accordance with this invention, an OCT probe device comprising the features as defined in the attached independent claim is provided; advantageous, preferred embodiments are defined in the dependent claims.

According to the invention, an OCT probe device, in particular a FD-OCT device, is provided which allows self-referenced interferometer topologies with simplified system construction and handling. In particular, the device may be fundamentally more compact and simpler to build in a tiny space than prior art devices. Problems of dispersion and polarization matching, as well as beam splitter spectral non-uniformity, are mitigated when the “interferometer” (signal and reference arm) is wholly contained in the endoscope tip portion.

According to an aspect of the invention, a common path approach is suggested where a reference reflection originating from the inside surface of the glass window is used. According to another aspect of the invention, an alternative approach is proposed which allows much more efficient collection of the reference reflection using a specific beam splitter design to achieve high speed in vivo ultra high axial resolution tomograms.

The FD-OCT system may use a compact mode-locked laser as light source, said laser emitting a broad spectrum, in combination with a photodetector array based, spectrally sensitive detector, as is known per se. Alternatively a spectrally scanning laser with single or multiple detectors may be implemented.

More in detail, the present concept of a distally integrated interferometer endoscope with optimized built-in components overcomes the most troublesome aspects of UHR (ultra high resolution)-OCT endoscopy. The present topology removes the need for a separate adjustable reference arm and therefore reduces system complexity and cost. There is no longer a need for tight tolerance on the length of the endoscope, potentially reducing the cost of this consumable element. No compensation is required when a new endoscope is attached to the system, allowing “plug-and-play” utility that is essential for widespread clinical use. System induced dispersion and polarization mismatch between sample and reference reflections is practically eliminated, allowing systems to achieve better resolution and sensitivity without dispersion or polarization compensating elements, and alignment time. Reduction in complexity shortens the troubleshooting process. It is to be expected that the beam splitters used are spectrally flat over a much wider range than the fiber beam splitters that are currently used in traditional Michelson interferometers. The spectral flatness of the present beam splitter configurations may be limited by the chromatic error in the focussing lens and the extent that the reference light is retroreflected into the spectrally dependant numerical aperture of a light carrying fiber. Swept source implementations may have no aligned optics outside the laser and have the potential to be extremely rugged.

The common path interferometer according to a first embodiment of the invention and using a reflection from the endoscope window as a reference is an extremely simple self-referenced solution, but image quality may be relatively low due to inefficient collection of the reference reflection. The image is stable relative to the window even if the mechanical tolerances of the endoscope allow the separation between the tip optics and protective wall envelope to vary. This stability improves image quality and may enable sensitive phase measurement. In tests, high signal amplification was required because the coupling of the unoptimized reference reflection was rather weak because of the focal offset inherently introduced by the optical path displacement in respect to the sample. Very good sensitivity is reached here when the interference term between the sample (signal) light and the reference light is maximized to fill as much of the dynamic range of the detector as possible. In practice with biological samples, which have inherently low backscattering, and where the illumination power is restricted either by the light source or the sample, one first attempts to get as much light back from the sample as possible. Then one usually tries to optimize the strength of the reference signal to fill the remaining dynamic range of the detector, which is a fixed ratio of the sampling light in the common path topology. The optimized reference power will generally be much stronger than the power returning from the sample. A single strong reference reflection improves the signal strength relative to the “autocorrelation signal”, and ghost reflections resulting from spurious “references” are relatively weak. The reflection of the window can be enhanced by adding a specifically highly reflecting (or backscattering) coating that will dominate over other weaker reflections. The obvious location preferably is the inner (or, possibly, the outer) surface of the window, although it is even conceivable to place this reflecting surface within the thickness of the window by placing a transparent jacket over the reflective coating. There are two significant problems with this solution which, nevertheless, would yet be workable for many applications. First, the coupling efficiency of the backreflected wave is low because the wavefront curvature does not match the shape of the window well, due to the displacement of the window away from focus. Lens design simulations predict that only few percent of the light reflected from the window is coupled back into the fiber. To achieve a strong reference signal, a highly reflective coating is required; then the interferometer might be inefficient in its collection of the sample beam. Second, if the reflection from one of the window surfaces is used as a reference, the reflection from the nearly parallel opposite surface of the window must be well suppressed in order to avoid a spurious reference. That is, e.g. about four percent reflection from an uncoated glass surface must be significant in comparison to an anti-reflective coating on the other glass surface. This is particularly important if one would use the outer surface as the reference.

According to another preferred embodiment, to optimize the strength of the reference reflection, a separate (short) beam path is introduced by means of a separate beam splitter behind the focussing lens. This embodiment of the present distally integrated interferometer shares many of the advantages of the common path topology because there is no fiber in the difference path, which is generally responsible for dispersion and polarization mismatch in endoscopic OCT. A wavefront matched radius on the reference reflector (mirror), as is preferred, allows efficient collection of the reflected beam and more than compensates for the theoretical efficiency advantage of a perfect common path arrangement. Changing the reflectivity of the beam splitter allows any ratio of sample power to reference power. The final choice for setting the beam splitter reflectivity depends on the source power available, the power that the sample can tolerate, the efficiency of the entire system, and the imaging speed desired. An adjustable cement spacer between the focussing lens, preferably a GRIN lens, and the beam splitter prism and or between the focussing lens and the fiber, proves to be a particularly advantageous element in the design to provide a compensator for manufacturing tolerances, by accordingly adjusting the thickness of this cement layer. Without this compensator, the radius of the reference mirror, the axial lengths of the focussing lens and the beam splitter prism, and the transmission of the beam splitter should all be specified rather tightly to achieve a reasonable yield of product with the expected collection of the reference beam. With a mature manufacturing process in a commercial environment, it is expected that tolerances are reduced enough to eliminate the need for this active alignment step. In the end, detector gain and imaging speed may be used as compensators to optimize system sensitivity. Changing the axial length of the second half of the beam splitter prism allows the reference to be placed at any depth, including beyond the endoscope window when a positive working distance is desired. The prism beam splitter also allows the flexibility to modify the dispersion of the reference arm to a small extent to compensate for water dispersion a short distance into the tissue.

This arrangement also allows the use of an intentionally off-normal beam exit angle to suppress unwanted backreflections, as an alternative to coatings or index matching. The added flexibility of this design comes at the cost of some loss in image stability.

Another preferred embodiment uses a split GRIN lens to split the beam at the end of the first element and focuses the reference beam with a further GRIN element onto a curved or planar reflector. The latter element might be replaced by non-GRIN material, which introduces a complex shape on the reflective surface for high throughput.

The “handicap” of limited freedom to adjust the reference arm in the common path interferometer is simultaneously one of its major advantages. Though +/− frequency ambiguity due to the real valued Fourier transform cannot be eliminated by modulation of the reference arm delay, transform mirror-artifacts do not appear because the zero delay point is inherently at a negative distance from the sample.

The invention will now be described in more detail with reference to the drawings where preferred exemplary embodiments of the invention are shown to which, of course, the invention should not be restricted. In particular,

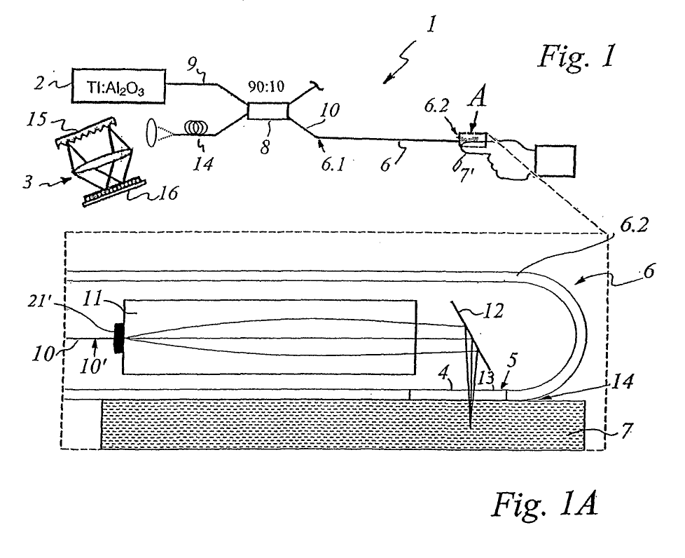

FIG. 1 diagrammatically shows a FD-OCT probe device coupled to a laser light source and to a detector;

FIG. 1A diagrammatically shows a cross-section of the tip portion of the endoscope, as referred to with “A” in FIG. 1, in an enlarged scale;

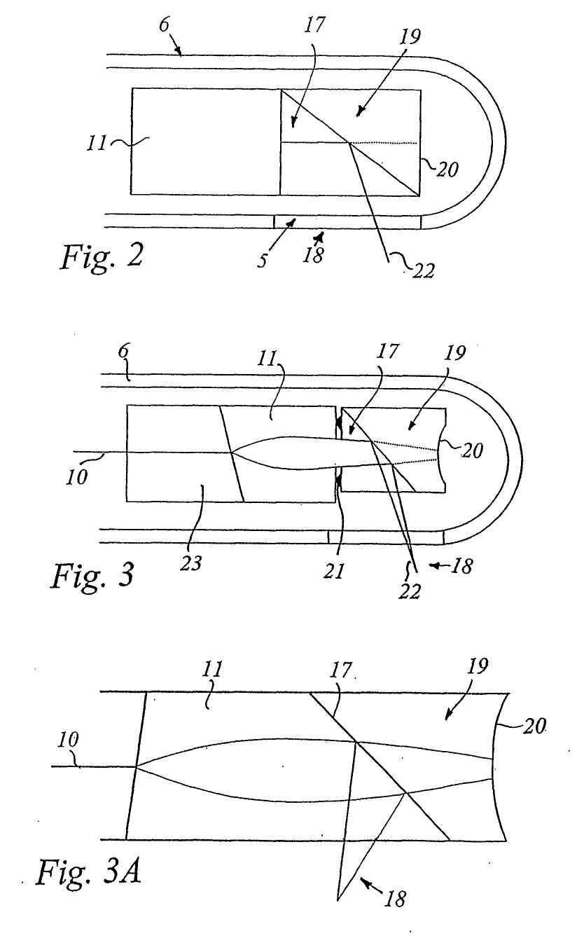

FIGS. 2, 3 and 3A show section views similar to FIG. 1A of modified interferometer embodiments;

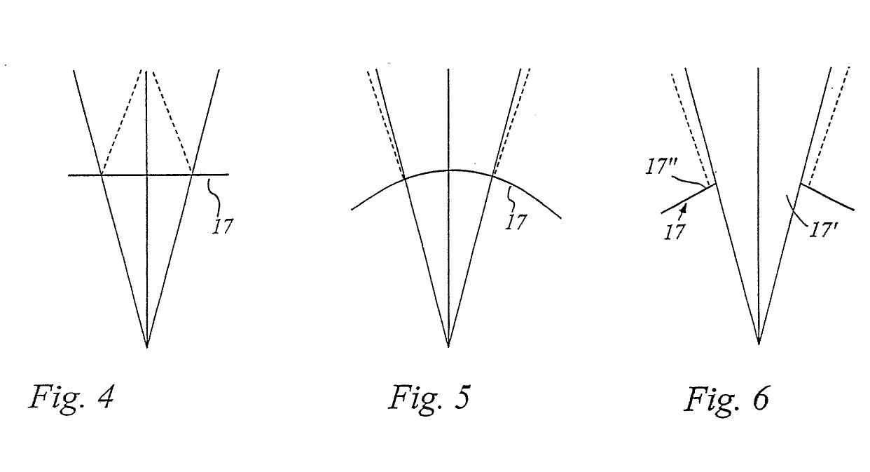

FIGS. 4 to 6 show quite schematic views of various beam splitter embodiments; and

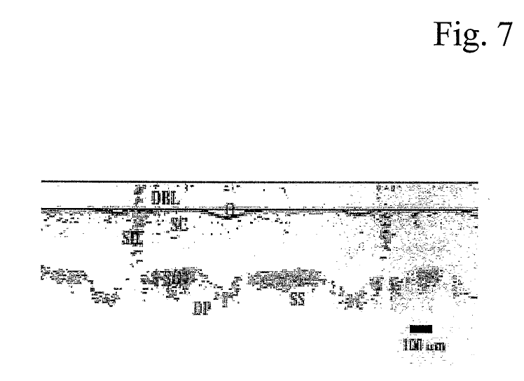

FIG. 7 shows a common path endoscopic ultra high resolution FD-OCT tomogram of a human fingertip tissue.

In FIG. 1 and FIG. 1A, there is shown a setup for a FD-OCT endoscope device 1 utilizing common path interferometer topology. Spatially resolved FD-OCT is achieved using a broad bandwidth laser light source 2 and a diffraction grating based spectrometer detector 3 yielding e.g. 2.9 μm axial resolution at 20,000 a-lines/s. The reference reflection originates at the inside surface 4 of a window 5 of the endoscope 6 proper, and is separated by the window thickness (e.g. 100 μm) from the subject to be scanned, namely from a tissue 7, in particular of a fingerskin 7′, compare FIG. 1.

More in detail, the light source 2 may be a compact femtosecond pulsed Ti:Sapphire (Ti:Al₂O₃) laser with 160 nm bandwidth at full-width-half-maximum (FWHM) centered at 800 nm for a theoretical axial resolution of 1.8 μm. For instance, a Femtosource Integral OCT laser commercially available from Femtolasers Produktions GmbH, Vienna, may be used. A 90/10 fiber beam splitter 8 is used to couple light from the laser light source 2 into the endoscope 6 and light from the endoscope 6 back to the detector 3. However, this fiber beam splitter 8 acts only as an optical coupler, i.e. as a spectrally flat optical circulator, and does not send a portion of the light beam which is received through fiber 9 to a pathlength matched reference arm, as has been provided for hitherto according to the prior art.

Light is transmitted then to the proximal portion 6.1 of the endoscope 6 and within the endoscope 6 by a fiber 10, so that an optical (light) path 101 is defined, and is focussed by a focussing lens, in particular a gradient index lens 11, and redirected by an air-spaced mirror 12 through the e.g. 100 μm thick fused silica window 5 into the tissue 7 at the distal tip portion 6.2 of the endoscope 6. The inner surface 4 of the window 5 acts as a thin beam splitter 13 of a common path interferometer built in within tip portion 6.2. A water-based lubricant 14 may be used for index matching at the interface between window 5 and finger-skin (tissue 7, and in particular to avoid an air gap at that interface). The dominant reference reflection comes from the inside surface 4 of the window 5. The window 5 introduces dispersion and resolution loss approximately equal to the same thickness of water.

The interference signal obtained by composing the sample light and the reference light in the tip portion 6.2 and returning from the endoscope 6 is directed into a spectrometer detector 3 consisting, in a manner known per se, of a polarization controller 14 to optimize transmission at a diffraction grating 15 focussed by a commercial camera objective onto a high speed linear CCD array 16 operating at e.g. 20,000 samples per second. The resolution of the CCD array 16 allows calculation to an optical depth of e.g. 1.4 mm, however limited spectrometer resolution causes a finite spectral bandwidth to be measured at each pixel and thereby may limit usable depth range to less than 1 mm. The axial pixel dimension after Fourier transform (the usual computer therefore not being shown in FIG. 1) may be about 1.34 μm. According to FIG. 2, the endoscopic arm 6 of the OCT device includes a distally integrated micro beam splitter 17. The beam splitter 17 receives the converging cone of light from the gradient index (GRIN) lens 11 and reflects 80% into the sample arm 18, transmits 10% to the reference arm 19 and absorbs about 10% of the incident light. An aluminum coated reference reflect- or (mirror) 20 is located so that, if all elements 11, 17, 20 remain perfectly centered in the endoscope 6, the reference will be located e.g. 100 μm in optical path length proximal to the endoscope outer envelope surface.

Preferably, the reference mirror 20 may be curved (also nonspherical) as shown in FIGS. 3 and 3A, to match the incident wavefront curvature for maximum fiber coupling efficiency of the return beam. Furthermore, according to FIG. 3, a thin cement gap 21 may serve as an alignment compensator that allows the part to be specified with generous tolerances and enables intentional attenuation of the reference beam without modifying the coating of the beam splitter 17. A similar optical cement layer 21′ (compare FIG. 1A) may be used to connect the optical fiber 10 to the focussing lens 11. Also here, the thickness of the cement layer 21′ may be adjusted to compensate for manufacturing tolerances.

A for instance 49° beam splitter angle (compare FIGS. 2, 3 and 3A) sends the output beam 22 off normal to exit surface of the beam splitter 17 and surfaces of the window 5 to suppress coupling of backreflections from these surfaces, which could cause spurious references. The difference in material path length from the solid thickness of the glass element (e.g. BK7) in the reference arm 19 and the airgap in the sample arm 18 causes primary dispersion from water to be corrected to a depth of e.g. 200 μm.

With respect to the gradient index (GRIN) focussing lens 11 as shown in FIGS. 1 to 3 and 3A, it may be added that it is well known in the art that such GRIN lenses operate on the principle of a continuous change of the refractive index within the lens material. Instead of complicated shaped surfaces, plane optical and surfaces can be used. In such a GRIN lens, the light rays are continuously bent within the lens until finally they are focussed on a spot. It is possible to fabricate miniaturized lenses down to 0.2 mm in thickness of diameter. The fiber mounting ferule, the GRIN lens, and the beam splitter prism may all be manufactured with an identical cylindrical outer diameter and be cemented to each other at their flat optical surfaces. The simple geometry allows a very cost-effective production and simplifies the assembly.

With specific respect to FIG. 3A, a preferred embodiment is shown where a combined lens and beam splitter arrangement is shown. More particularly, a GRIN focussing lens 11 is directly combined with a beam splitter element 17 where the beam is partly reflected at the interface to the lens 11, and is partly transmitted, as reference beam, to a planar or preferably curved reflector 20. The latter element 17 may be a GRIN element, but may be comprised of non-GRIN material, too.

Then, with respect to FIGS. 4 to 6, several reflection situations are shown very schematically. First, according to FIG. 4, the situation as appearing with the FIG. 2 embodiment at the reference mirror 20 is shown. It can be seen that the beam splitter 17 is a partially reflective element which sends a fraction of the entire beam back at an angle twice the angle of incidence on the mirror surface.

Therefore, to get high efficiency, the surface of the mirror 17 (compare FIG. 3) may be matched to the wavefront shape at that point; this usually requires as spherical surface, as is shown in FIG. 5. Here, the beam travels back more closely along the beam path.

Instead of these cases of specular reflection, also the principle of special sampling may be applied, compare the mirror 17 of FIG. 6. Namely, it is not necessary to sample the entire beam. Instead, it is possible to use a highly reflecting mirror segment 17″ which may only sample a portion of it, allowing the rest of the beam to hit the target by passing another segment 17′.

It should be mentioned that moreover, a subset of a scattering beam splitter may be used, where surfaces can be manufactured on planar materials that specifically alter the phase of the backscattered wavefront so that it may be collected with high efficiency (not shown).

In a test setup, the lateral scan was performed at 14.3 mm/s corresponding to a sampling density of 1400 a-lines/mm. The 2 mm outer diameter endoscope 6 achieved a lateral scan by pushing and pulling the tip optics (compare FIG. 1A) in usual manner via mechanical linkage (not shown) to an external stepper motor driven linear actuator (not shown). Tomograms were recorded from the subject to be scanned, here the in vivo normal human fingertip skin, at 3 mW incident power.

System performance is quantified by examining resulting tomograms. Axial resolution is measured from the specular reflection originating from the outer surface of the window 5. The dynamic range is calculated from the maximum valued pixel in each a-line, excluding the top 200 pixels which contain the specular reflections from the outer window and skin surfaces, and comparing that to the standard deviation of the noise in an area near the bottom of the image.

In a specific test of the device described above and with reference to FIG. 1; 1A using the common path approach, a human fingertip skin was scanned which exhibited high stability and axial resolution (2.9 μm). As may be seen from FIG. 7, image features included stratum corneum SC, stratum granulosum SG, sweat duct SD, and possibly dermal papillae DP reaching up into the stratum spinosum SS, the outer surface of endoscope window O, and a faint double image DBL of stratum corneum in view of the endoscope window outer surface acting as a reference.

The in vivo tomogram of the ventral surface of a human fingertip showed that high axial resolution (corresponding to 4.0 μm in air or 2.9 μm in tissue with index=1.4) has been achieved with no effort to match pathlength, dispersion, or polarization between sample and reference arms. The tomogram exhibited an average dynamic range of 27 dB (38 dB max), with 3 mW incident on the tissue. The tomogram was displayed with dimensions corrected for an average tissue index of 1.4. Sweat ducts were clearly resolved in the stratum corneum, and penetration reached slightly below the stratum granulosum, approximately 0.5 mm into the tissue. A lack of signal at the bottom of the tomogram was characteristic of the dermal papillae reaching up into the stratum spinosum, but could also easily be confused with signal falloff at this depth. The reflection from the outer surface of the endoscope window 5 was observed as a thin line, frequently in contact with tissue 7, 145 μm in optical thickness from the top of the image. The outer surface of the window 5, which was relatively close to the beam focus, would present a stronger back reflection than the inside surface, but did not because the lubricant 14 provided efficient index matching to the glass of the window 5. A faint double image displaced ˜100 μm, or 145 μm in optical thickness, vertically was the result of this second “reference” reflection coming from the outer surface of the endoscope window. This double image was noticeable at the top of the image space “inside” the window.

For testing the device described with reference to FIG. 3, normal mice, approximately ten weeks old, were imaged in vivo to demonstrate the potential of the technique to image disease model mice. Mice were anesthetized with a mix of Ketamine-Xylazine delivered with an intramuscular injection. The endoscope was coated with a water based lubricant and inserted in the anus to a depth of 33 mm. Longitudinal tomograms were collected at 14.3 mm/s with 3 mW power incident on the sample. System performance was quantified as described above for the common path configuration.

1-20. (canceled)

21. An optical coherence tomography probe device, comprising:
endoscope means having a first, proximal portion adapted to be coupled to a light source through optical coupling means, and a second, distal tip portion;
said endoscope means defining a light path in an interior thereof for transmitting light emitted from the light source and coupled into said proximal portion to said tip portion;
said tip portion including focusing lens means and a window for directing light to an object to be scanned, for receiving light scattered at the object, for transmitting the scattered light back through said endoscope means and to said optical coupling means, and wherein said optical coupling means are further adapted to apply the scattered light together with reference light to an interferometric detector;
a beam splitter configured to separate the reference light from remaining light scattered at the object, and a reference light reflector disposed to reflect the reference light back to form composite light with the light returned from the object, to obtain an interference signal by said interferometric detector; and
wherein said beam splitter and said reference light reflector are disposed in said tip portion of said endoscope means behind said focusing lens means through which said composed light is sent back along a common light path within said endoscope means to said optical coupling means.

22. The OCT probe device according to claim 21, wherein said beam splitter and said reference light reflector are, in combination, formed by said window, and said window has a surface configured to partly reflect light.

23. The OCT probe device according to claim 22, wherein the window comprises a surface having a partly reflective coating made up with at least one material selected from the group of materials including Al, Ag and a dielectric stack material.

24. The OCT probe device according to claim 23, wherein said surface having a partly reflective coating is formed by an inside surface of said window.

25. The OCT probe device according to claim 21, wherein said beam splitter is a partly reflective beam splitter prism disposed within said tip portion of said endoscope means and adjacent said window, and said reference light reflector is located behind said beam splitter prism, to thereby define a short reference arm.

26. The OCT probe device according to claim 25, which comprises an optical cement layer disposed to connect said beam splitter prism to said focusing lens means.

27. The OCT probe device according to claim 21, wherein said focusing lens means is connected to an optical fiber by way of an optical cement layer, and said optical fiber defines said light path.

28. The OCT probe device according to claim 26, wherein said optical cement layer has a thickness adjusted as a compensator for manufacturing tolerances.

29. The OCT probe device according to claim 25, wherein said beam splitter prism is formed with a partly reflecting surface inclined with respect to a main axis of an impinging light under an angle diverting from 45°, to eliminate interferences with possible light reflections from said window.

30. The OCT probe device according to claim 25, wherein said reference light reflector is formed with a curved reflecting surface, for matching to a shape of a light wave front at said reflecting surface.

31. The OCT probe device according to claim 21, wherein said focusing lens means is a cylindrically shaped gradient index lens.

32. The OCT probe device according to claim 25, wherein said focusing lens means and said beam splitter have substantially identical cylindrical diameters.

33. The OCT probe device according to claim 25, which further comprises fiber mounting means having a substantially identical cylindrical diameter as said focusing lens means and said beam splitter.

34. The OCT probe device according to claim 25, wherein an amount of optical dispersion present in the reference arm compensates for a dispersion of water a predetermined distance into the object.

35. The OCT probe device according to claim 25, wherein said reference reflector surface is placed at a distance corresponding to a location substantially equivalent to an exterior surface of said endoscope window.

36. The OCT probe device according to claim 25, wherein said reference reflector surface is placed at a distance corresponding to a location substantially beyond an exterior surface of said endoscope window.

37. The OCT probe device according to claim 21, wherein said optical coupling means is an optical fiber beam splitter configured for coupling a minor portion of light emitted by the light source to said endoscope means but for coupling a major part of the light coming back through said endoscope means to said detector.

38. The OCT probe device according to claim 37, wherein the minor portion is approximately 10% of the light emitted by the light source and the major portion is approximately 90% of the light coming back through said endoscope means.

39. The OCT probe device according to claim 21, wherein said endoscope means comprises an optical fiber for transmission of light between the optical coupling means and the focusing lens means.

40. The OCT probe device according to claim 21, which further comprises an index-matching lubricant applied to an outer window surface for avoiding an air gap there.

41. The OCT probe device according to claim 21, wherein said beam splitter has a segment allowing a portion of a light beam to pass through and a reflecting segment for highly reflecting a rest of the beam.

42. A method of adjusting an optical coherence tomography probe device during a manufacture thereof, the method which comprises:
assembling endoscope means having a first, proximal portion adapted to be coupled to a light source through optical coupling means, and a second, distal tip portion, the endoscope means defining a light path in an interior thereof for transmitting light emitted from the light source and coupled into the proximal portion to the tip portion; the tip portion including focusing lens means and a window for directing light to an object to be scanned, for receiving light scattered at the object, for transmitting the scattered light back through the endoscope means and to the optical coupling means, and wherein said optical coupling means are further adapted to apply the scattered light together with reference light to an interferometric detector; a partly reflective beam splitter prism configured to separate the reference light from remaining light scattered at the object, and a reference light reflector disposed to reflect the reference light back to form composite light with the light returned from the object, to obtain an interference signal by the interferometric detector; and placing the reference light reflector behind the beam splitter prism in the tip portion of the endoscope means, to thereby form a short reference arm; and
connecting the beam splitter prism to the focusing lens means with an optical cement layer and adjusting a thickness of the optical cement layer to form a compensator for manufacturing tolerances.

43. A method of adjusting an optical coherence tomography probe device during a manufacture thereof, the method which comprises:
assembling endoscope means having a first, proximal portion adapted to be coupled to a light source through optical coupling means, and a second, distal tip portion, an optical fiber defining a light path in an interior of the endoscope means for transmitting light emitted from the light source and coupled into the proximal portion to the tip portion; the tip portion including focusing lens means and a window for directing light to an object to be scanned, for receiving light scattered at the object, for transmitting the scattered light back through the endoscope means and to the optical coupling means, and wherein said optical coupling means are further adapted to apply the scattered light together with reference light to an interferometric detector; a beam splitter configured to separate the reference light from remaining light scattered at the object, and a reference light reflector disposed to reflect the reference light back to form composite light with the light returned from the object, to obtain an interference signal by the interferometric detector; and
connecting the optical fiber defining the light path to the focusing lens means with an optical cement layer and adjusting a thickness of the optical cement layer to form a compensator for manufacturing tolerances.
